(19) United States
(12) Patent Application Publication
(10) Pub. No.: US 2016/0193123 A1
(43) Pub. Date: Jul. 7, 2016

(54) AQUEOUS SUSPENSION OF NANOCAPSULES ENCAPSULATING SUN FILTERS

(71) Applicants: POLAAR, Paris (FR); UNIVERSITE CLAUDE BERNARD, Villeurbanne (FR)

(72) Inventors: Aurélie DE LARMINAT, Bourg la Reine (FR); Daniel KURBIEL, Chaville (FR); Fabrice PIROT, Lyon (FR); Elodie GILBERT, Villeurbanne (FR)

(73) Assignees: POLAAR, Paris (FR); UNIVERSITE CLAUDE BERNARD, Villeurbanne (FR)

(21) Appl. No.: 14/911,922

(22) PCT Filed: Aug. 13, 2014
(86) PCT No.: PCT/FR2014/052086

§ 371 Date: Feb. 12, 2016

(30) Foreign Application Priority Data
Aug. 13, 2013 (FR) 13/57987

(51) Int. Cl. A61K 8/11; A61K 8/92; A61Q 17/04; A61K 8/29; A61K 8/35; A61K 8/04; A61K 8/85

(52) U.S. Cl. CPC A61K 8/11; A61K 8/044; A61K 8/92; A61K 8/85; A61K 8/29; A61K 8/35; A61Q 17/04; A61K 2800/56; A61K 2800/413

(57) ABSTRACT

An aqueous suspension of nanocapsules encapsulating at least one UV filter which is likely to be obtained by a preparation process in which are mixed: (a) a first phase, called oily phase, which includes: at least one hydrophobic polymer, at least one oil, at least one UV filter, and at least a first surfactant; (b) a second phase, called aqueous phase, which includes water and/or at least one polar solvent, and optionally at least one second surfactant. The object is also a sunscreen composition including this suspension.

The present invention concerns the field of the formulations of sunscreens.

The sunscreens comprise specific molecules which are the ultraviolet filters (hereinafter abbreviated UV). The most effective UV filters protect against:
UV-A rays which are rays with wavelength comprised between 320 and 400 nm,
UV-B rays which are rays with wavelength comprised between 280 and 320 nm.

Indeed, these UV rays are harmful to the skin. The rays with the shortest wavelengths are the most aggressive and those with the longest wavelengths penetrate more deeply into the skin. The UV-A rays are responsible for an immediate pigmentation of the skin, but also of its premature ageing, of an immunosuppression and of skin cancers. The UV-B rays are responsible for the synthesis of vitamin D and the suntan (delayed pigmentation), but also for sunburns, of the immunosuppression and of skin cancers.

The sunscreens are characterized by their sun protection factor (hereinafter abbreviated SPF).

The SPF is defined according to the following formula (I):

$$S P F = \frac{M E D on protected skin}{M E D on unprotected skin} \qquad ( I )$$

MED is the abbreviation of [symbol] Minimal Erythema Dose [symbol] .

For that a sunscreen has optimal efficiency, it must meet the following criteria:
maintaining of the UV filters to the skin surface;
protection in the UVB and UVA spectra;
photostable.

The formulation of a sunscreen is dependent on the physicochemical properties of the incorporated UV filters.

The UV filters are classified into two categories: the organic UV filters and the mineral UV filters.

The organic UV filters (also called chemical UV filters) are organic molecules absorbing and dissipating the UV rays by chemical reactions. Most of these organic UV filters are lipophilic. Their maximum concentrations and their combinations between each other in sunscreen formulations are perfectly regulated.

By way of example of organic UV filters, oxybenzone, octrocrylene, avobenzone, octyl methoxycinnamate may be mentioned.

The organic UV filters have the advantages of being easily incorporated into non-pasty sunscreen formulations and pleasant for the user.

However, the organic UV filters are also known by their photo-instability (for example avobenzone), their allergenic and polluting character, their tendency to cross the skin barrier (that is to say, the stratum corneum) and finally to some, their endocrine disturbing properties (for example oxybenzone).

There are also the mineral UV filters which are mineral particles reflecting and absorbing the UVA and the UVB. These mineral particles are usually covered with a hydrophilic or hydrophobic coating (for example made of methoxysilane, dimethicone, silica or alumina) which inhibits their photo-reactivity and facilitates their incorporation into the sunscreen formulations.

By way of example of mineral UV filters, titanium dioxide, zinc oxide, kaolin and talc may be mentioned.

The mineral UV filters exhibit the advantages of being hypoallergenic and not crossing the skin barrier.

However, they are proving to be more difficult to be formulated than the organic UV filters, because the sunscreen formulations in which these UV filters are incorporated, are often thicker and tend to leave unsightly white marks on the skin.

In order to overcome the problem of these white marks, it could be possible to reduce the concentration of these minerals UV filters. However, this solution would not be satisfactory, because it would decrease the sunscreen protection. But that would go against one of the objectives to be reached by sunscreen formulations which are ensuring a maximum sun protection.

Finally, there are natural molecules which have intrinsically the property of filtering the UV. They can therefore be used as UV filters, alone or as a complement to organic or mineral UV filters. For example carnauba wax, olive oil, karanja oil, usnic acid, propolis, cucumber extract, polyphenols may be mentioned.

The sunscreens may be provided in different formulation types, among which the fluid (milks) or thick (creams) emulsions; the gel- or oil-type formulations; the sticks formulations; the lotions may be mentioned.

When the sunscreen is an emulsion, it comprises a lipid phase, an aqueous phase and one or more surfactant(s).

The UV filters are dispersed:
in the lipid phase if they are lipophilic
in the aqueous phase if they are hydrophilic.

When the sunscreen is in the form of an oil, it is about a lipid phase in which lipophilic UV filters was dispersed.

The hydrophobic sun gels result from the dispersion of lipophilic UV filters in a lipid phase which was then gelled through adding a gelling agent. The hydrophilic sun gels result from the dispersion of hydrophilic UV filters in an aqueous phase which was then gelled through adding a gelling agent.

Finally, sun sticks are manufactured from a lipid phase comprising wax in which lipophilic UV filter was dispersed. The thus obtained mixture was cooled in molds giving a stick shape to the sunscreen formulation.

Also, it is known adding excipients to the sunscreen formulations in order to ensure an optimal spreading of the UV filters on the skin surface by forming an homogeneous protective film to be applied.

In this regard, the passage of the UV filters through the skin may be promoted by the following factors:
poor general condition of the skin;
the disorganization of the lipids and the tight junctions of the skin barrier under the action of the UV rays;
some solvents such as ethanol, propylene glycol, terpenes;
some emollient agents;
their molecular weight (the molecules whose molecular weights is lower than 500 Da are more likely to cross the skin barrier than the molecules with greater molecular weight).

The organic UV filters are, mostly lipophilic molecules with low molecular weight. They are therefore likely to cross the skin barrier and thus to reach the nucleated cells of the skin, then the systemic circulation which may be very prejudicial for the user of the sunscreen.

This is why scientific developments on the sunscreen formulations was carried out to [symbol] trap [symbol] the UV filter in a particle, so that it is maintained on the surface of the skin, and thus to prevent its transcutaneous passage, to overcome the problems of photo-instability, irritation, photo-allergic and photo-toxic reactions and to improve its filtering capability.

These researches have led to the design of new vehicle (or in other words, of carriers) of UV filters, in order to improve the performances of the sunscreen formulations.

The use of multi-particulate systems trapping UV filters aroused a significant interest. Indeed, besides the advantage of being easily incorporated into the customary sunscreen formulations which was detailed hereinabove, these multi-particulate systems may have the ability to absorb and/or to reflect UV radiation thus acting on their own as physical sunscreens.

Among these multi-particulate systems there are firstly the solid lipid nanoparticles (hereinafter abbreviated [symbol] SLN [symbol] ), it is about oily droplets of lipid solids at the body temperature which are stabilized by surfactants. In other words, SLN are nanoparticles constituted of a solid lipid core enveloped by one or more surfactant(s) suspended in an aqueous phase.

The SLN have occlusive properties ideal for the cosmetic products for sun protection. Studies have shown that formulations containing SLN in which UV filters was encapsulated exhibited an improved filtering capability in the UV spectrum.

In this regard, there is known the application US 2003/0235540 A1 which describes SLN compositions containing liposoluble UV filters. It is stated that due to these compositions, the penetration into the skin of the encapsulated UV filters is reduced, having thereby a positive impact on the toxicity problems posed by some UV filters.

However, the SLN exhibit the following disadvantages:
the amount of UV filters which may be encapsulated are limited;
the UV filters tend to be expelled out of the nanoparticle during the storage of the sunscreen formulation.

These instability problems are inherent to the solid lipids constituting the matrix of the SLN which tend to form a perfect crystal lattice whose interstices make it possible to eject the UV filters out of the nanoparticle. The SLN may be more or less sensitive to heat depending on the melting point of the used solid lipid matrix. If the solid lipid matrix comes to melt, this may disorganize the system; which may cause a decrease in the filtering capability of the SLN in the UV but also the total phase shift of the SLN suspension.

Finally, the significant presence of solid lipid whose melting point is greater than the skin temperature (that is to say greater than 32° C.) makes the SLN sunscreen formulations difficult to be spread; which is not suitable for a sun protection product which must be easily and uniformly spread on the skin.

Thus, in the current state of knowledge on the properties of the SLN, the sunscreen formulations comprising encapsulated UV filters with this system does not prove to be entirely satisfactory.

In order to overcome the disadvantages inherent to the SLN, another trapping system of UV filters was developed which consists of lipid particles resulting from the solid lipids mixture with liquid lipids. These particles are known as nanostructured lipid carriers (hereinafter abbreviated [symbol] NLC [symbol] ). In other words, the NLC are nanoparticles consisting of a solid and liquid lipid core enveloped by one or more surfactant(s) suspended in an aqueous phase.

Compared to the SLN, NLC have a heterogeneous structure which gives an imperfect structure to their matrix having spaces in which the UV filters may be housed. This allows overcoming the ejection problems of the UV filters encountered with the SLN. But, the NLC, due to the presence of solid lipids, have the same defect type of spreading and heat sensitivity than the SLN.

Although their most significant encapsulating capability of UV filters and their best stability make the NLC more suitable than the SLN in the field of the sunscreen formulation, it may be quite advantageous to implement other encapsulating systems of UV filter exhibiting improved sun protection performances, and this, in particular by seeking to optimize the amounts of encapsulated UV filters.

Thus, as explained hereinabove, we are always looking for sunscreen formulations exhibiting the best possible sun protection performances, and this, by reconciling the following parameters which are:
the good spreading of the UV filters on the skin,
prevention of the transcutaneous passage of the UV filters,
the improved aesthetic properties such as the mitigation of white marks left on the skin by the mineral UV filters after application.

Furthermore, the application WO 2010/040194 A2 which describes polymer nanocapsules containing oil and a UV filter, as well as their manufacturing process, is known.

The manufacturing process described in this international application systematically uses organic solvents and implements a technique selected from multiple techniques such as the in situ polymerization of dispersed monomers, the emulsion, interfacial polymerization, the precipitation of preformed polymers, the nanoprecipitation, the interfacial deposition, the emulsification-evaporation or even the emulsification-diffusion. In this regard, the only manufacturing example of nanocapsules described in this international application uses the interfacial deposition technique. The solvent is acetone and is evaporated at the end of the process.

However, the use of an organic solvent in the oily phase during the manufacturing of nanocapsules described in this international application WO 2010/040194 A2 has the disadvantage that the final product may comprise traces of this organic solvent, which is absolutely to be avoided. Furthermore, the evaporation step of the organic solvent is constraining, difficult to be implemented and lengthens the period of manufacture of said nanocapsules. Thus, this considerably reduces the industrial interest of these nanocapsules including a UV filter.

That is why, as opposed to the application WO 2010/040194 A2, it would be advantageous to develop a process for manufacturing nanocapsules with an oily core encapsulating a UV filter and which is free of any organic solvent such as ethyl acetate and acetone which are solvents conventionally used as preparation adjuvant to dissolve the hydrophobic polymers in an oily phase, and while obtaining nanocapsules having excellent sun protection performances.

Furthermore, the French patent application FR 2 930 176 A1 describing nanocapsules used as carrier agents of active ingredients which ensure a good protection of the encapsulated active ingredient as well as a sustained and/or controlled release thereof in vivo. These nanocapsules are used to convey pharmaceutical active ingredients, such as for example chlorhexidine base, minoxidil, albendazole and ketoconazole.

However, the inventors of the present invention, which are also the inventors of this French patent application FR 2 930 176 A1, have discovered, surprisingly, that the encapsulation of UV filters in nanocapsules comprising an oily core surrounded by a polymeric envelope prepared according to a process being close, by some technical aspects, to that described in this French patent application allow obtaining sunscreen formulations having excellent in vitro sun protection performances, and in particular obviously better than those obtained with sunscreen formulations in which these same UV filters were not encapsulated.

The present invention firstly provides an aqueous suspension of nanocapsules which comprise an oily core wherein at least one UV filter is homogeneously dispersed and a polymeric envelope containing at least one hydrophobic polymer, said aqueous suspension of nanocapsules is likely to be obtained by a preparation process in which are mixed:

a) a first phase, said oily phase which includes:
at least one hydrophobic polymer,
at least one oil,
at least one UV filter, and
at least a first surfactant,
the first phase being brought to a temperature T1 greater than the melting temperature of the hydrophobic polymer,
at this temperature T1, the hydrophobic polymer being miscible with the mixture of the first surfactant and the oil, and the UV filter is miscible, soluble or solubilized, in the mixture of the first surfactant and of the oil;

b) a second phase, called aqueous phase, which comprises water and/or at least one polar solvent, and optionally at least one second surfactant,

so as to obtain the formation of nanocapsules in aqueous suspension.

The aqueous suspension thus obtained is in the form of a milky homogeneous mixture.

The nanocapsules thus formed comprise an oily core in which at least one UV filter is homogeneously dispersed and a polymeric envelope containing at least one hydrophobic polymer.

The UV filter may be encapsulated in the oily core of the nanocapsules, or adsorbed within the polymeric envelope. More specifically, the UV filter may be mostly trapped within the oily core of the nanocapsules and the remaining part of the UV filter is adsorbed on the polymeric envelope containing the hydrophobic polymer or the UV filter may be partially trapped in the oily core and partially adsorbed onto said polymeric envelope or even the UV filter may be mostly adsorbed on the polymeric envelope and the remaining part of the UV filter is trapped in the oily core.

Thus, the aqueous suspension of nanocapsules according to the invention always comprises at least one part of the UV filter which is homogeneously dispersed in the oily core of said nanocapsules.

The nanocapsules have a diameter lower than 1000 nm, preferably comprised between 100 and 700 nm.

The aqueous suspension of nanocapsules thus obtained may be diluted with water, without significant change of the stability of the suspension.

The stability of these nanocapsules is proven. They allow protecting the UV filter(s) encapsulated in their core or adsorbed within the polymeric envelope of the degradation phenomena.

The aqueous suspension thus obtained may then either be used as such as an ingredient in a sunscreen formulation, or lyophilized before being incorporated as an active ingredient in a formulation. This latter option will be preferred for lipophilic formulations of the oil and stick types in which the incorporation of water is difficult, or even impossible.

More specifically, the aqueous suspensions of nanocapsules obtained according to the invention may usefully be freeze-dried as known from the prior art and then perfectly within the capabilities of those skilled in the art. Conventionally, the suspensions are pre-freezed at a temperature of −80° C. and are placed in a freeze dryer in which the temperature is close to −55° C. with a significant vacuum. The lyophilizate thus obtained can be sifted and redispersed in an aqueous solution.

The present invention has another object of a sunscreen composition comprising at least one aqueous suspension of nanocapsules according to the invention as described hereinabove.

Preferably, said sunscreen composition further comprises at least one physiologically acceptable excipient.

This excipient is advantageously selected from the excipients usually used in the sunscreen formulations, among which it can be mentioned: thickening texture agents (for example xanthan gum, guar gum, alginates); texture emulsifying agents forming a uniform and uninterrupted film on the skin; the film-forming agents providing a uniform protective film and increasing the water resistance; the moisturizing agents (for example glycerin) which retain water in the skin; the soothing agents (for example allantoin) for their healing and regenerating effects; the fragrances; the pigments; the conservatives for their inhibiting capability of the microbial growth (for example sodium benzoate, potassium sorbate, parabens).

Preferably, the excipients will be selected to prevent the passage of the UV filters through the skin barrier.

The sunscreen composition according to the invention may be formulated in the form of a fluid emulsion, such as a milk or a thick emulsion such as a cream, a gel, an oil, or even a stick or a lotion.

Preferably, the sunscreen composition is formulated in the form of an emulsion, when it is intended to be used for a cosmetic application.

Thus, the aqueous suspension of nanocapsules according to the invention may be used as sun protection product, inter alia for:
cosmetic applications: protection of the skin and the hair against the harmful effects of the UV (including hair dyes, protection of the color);
industrial applications: protection of paints, varnishes, stains and oils for applications in the technical fields of construction, textiles, or of the packaging (for example, when the packaging is transparent, it may be necessary to protect the ingredients contained therein from the sun to avoid distorting their formulation).

Some definitions of used terms in the context of the description of the invention are given hereinafter.

The concepts of solubility, miscibility and solubilization are well known to those skilled in the art. Unless otherwise indicated, in the context of the invention, the solubility, miscibility or solubilization is obtained at room temperature, namely at about 20° C.

In particular, in the context of the invention, [symbol] miscible [symbol] a means completely miscible. Two liquid compounds will be considered when completely miscible when mixed together in any proportion. Accordingly, the term miscibility refers to the mutual solubility of the compounds in the liquid systems.

Within the scope of the invention, a solid compound will be considered as soluble in a liquid or mixture of liquids when this compound is homogeneously dispersed in the molecular state under the effect of spontaneous solid/liquid interactions.

Concerning the solubilization, a solid or liquid compound (mineral or organic) will be considered solubilized in a liquid or a mixture of liquids, in particular when a combination of micelle-forming colloids increases the solubility of the compound initially insoluble in the dispersion medium.

[symbol] Hydrophobic [symbol] polymer means a polymer insoluble in water.

UV filter means any molecule whose main or secondary property is to absorb the UV at a wavelength range comprised between 290 and 400 nm (UVB and UVA); which includes the chemical UV filters, the mineral screens, as well as other natural molecules or oils which have filtering properties in the UV (such as for example carnauba wax, olive oil, karanja oil, usnic acid, propolis, cucumber extract, polyphenols).

[symbol] Oil [symbol] means a lipophilic fat, which is non-miscible in water or slightly miscible in water. In the context of the present invention, it may be about an oil taken alone or in mixture. In other words, in the following description, [symbol] oil [symbol] means an oil or a mixture of liquid or solid oils.

[symbol] Water-dispersible oil [symbol] means an oil which is dispersed in water in the molecular, colloidal or micrometer state.

The HLB (hydrophilic lipophilic balance) will be determined by the Griffin's method. (Griffin W C: Classification of Surface-Active Agents by ‘HLB’, Journal of the Society of Cosmetic Chemists 1 (1949): 311. Griffin W C: Calculation of HLB Values of Non-Ionic Surfactants, Journal of the Society of Cosmetic Chemists 5 (1954): 259).

The diameter of the nanocapsules which corresponds to the largest dimension of the nanocapsules will be determined by particle size analysis.

In the context of the invention, the first and the second phases, respectively, said oily phase and aqueous phase, are mixed to result in the spontaneous formation of the nanocapsules. The percentages given hereinafter correspond:
concerning the oily phase, the mass percentage of each component, based on the total mass of the oily phase,
concerning the aqueous phase, to the mass percentage of each component based on the total mass of the aqueous phase.

The oily phase is homogeneous.

The oil(s) contained in this oily phase are hydrophobic by nature, and may in some cases be water dispersible. This oil or mixture of oils is intended to form the core of the nanocapsules. In particular, the oil or the mixture of oils may have a HLB of 5 comprised in the range from 3 to 6.

By way of example of oil which may be used in the context of the invention, it may be mentioned triglycerides, in particular medium chain triglycerides, propylene glycol dicaprylocaprates, macrogolglycerides oleoyles, lauroyles and linoleoyles, vegetable and animal waxes and vegetable oils. For example concerning waxes, it may be about rice wax, carnauba wax.

In a most preferred manner, the oil of the oily phase is carnauba wax.

The oily phase may comprise from 5% to 85% by mass of oil. Preferably, the oily phase comprises from 10% to 40% by mass of oil, even more preferably from 10% to 20% by mass of oil.

According to a variant of the invention, the oily phase comprises from 45% to 55% by mass of oil.

Of course, this percentage applies only to oil (or if applicable the mixture of oils) which does particularly not comprise UV filter and/or the first surfactant, even when the latters are also in the oily form.

The oily phase contains at least one hydrophobic polymer in the molten state, the oily phase being maintained at a temperature T1 greater than the melting temperature of the polymer.

The temperature T1 is appropriately selected so that the oily phase described hereinabove is homogeneous, that is to say that there are no solid particles within the oily phase.

In other words, the temperature T1 is appropriately selected so that it has complete melting of the components of the oily phase, and so that they are homogeneously mixed together. In particular, the temperature T1 is greater than the melting temperature of the hydrophobic polymer, during the mixing of the components of the oily phase, the hydrophobic polymer will be melted and will be mixed thoroughly with the other components of the oily phase.

It is essential that the temperature T1 is appropriately chosen so as to avoid degrading the components of the oily phase. Those skilled in the art knowing the melting temperatures of the components of the oily phase, will perfectly choose the temperature T1 depending on said components of the oily phase so as not to degrade it when preparing said oily phase.

In one embodiment of the invention, the temperature T1 is greater by about 5° C. and 10° C. than the melting temperature of the component of the oily phase having the lowest melting temperature, so as the components of the oily phase do not degrade.

In one embodiment of the invention, the temperature T1 is greater by about 10° C., preferably by about 5° C., than the melting temperature of the hydrophobic polymer. Thus, the mixture of the oily phase is homogeneous.

The hydrophobic polymer will be selected so that its melting temperature is compatible with the physicochemical stability of the oil, of the UV filter and of the first surfactant.

For example, the hydrophobic polymer may have a melting temperature lower than or equal to 120° C.

The hydrophobic polymer may be selected from vinyl polymers, polyesters, polyamides, polyurethanes, polycarbonates, preferably having a melting temperature lower than 120° C., such as polycaprolactones (such as for example poly-e-caprolactones).

The oily phase may comprise from 0.1% to 4% by mass, preferably from 0.1% to 0.5% by mass of hydrophobic polymer.

According to a variant of the invention, the oily phase comprises from 0.4% to 1% by mass of hydrophobic polymer.

The oily phase also contains at least one UV filter which is dispersed, in a miscible, soluble or solubilized form therein.

The UV filter is miscible, soluble or solubilized in the mixture composed of the first surfactant and the oil at the temperature T1.

According to a variant, the UV filter is also miscible, soluble or solubilized in the mixture composed of the first surfactant and the oil or the mixture of oils, at room temperature, in particular at 20° C.

When the UV filter is solubilized, its solubilization is carried out by the action of the first surfactant, acting as a solubilizing agent.

The UV filter may be selected from organic UV filters, the mineral UV filters or any compound having a filtering capability in the UVB and/or UVA.

Advantageously, the UV filter is selected from the group consisting of:
the organic UV filters absorbing the UV-A such as oxybenzone, sulisobenzone, dioxybenzone, ethyl anthranilate, avobenzone, terphatylidene, dicamphor sulfonic acid and bis-ethylhexyloxyphenol methoxyphenyl triazine;
the organic UV filters absorbing th UV-B such as para-aminobenzoic acid, amyl p-dimethyl para-aminobenzoic acid, 2-ethoxyethyl-p-methoxycinnamate, digalloyl trioleate, ethyl 4-bishydroxypropylaminobenzone, 2-ethoxyethyl 2-cyano-3,3,diphenylacrylate, 2-ethylhexyl-p-methoxycinnamate, 2-ethylhexyl salicylate, glyceryl para-aminobenzoic acid, homo-methyl salicylate, dihydroxyacetone, octyl dimethyl para-aminobenzoic acid, 2-phenylbenzimidazole sulfonic acid and triethanolamine salicylate;
the mineral UV filters such as titanium dioxide, zinc oxide, cerium oxide, kaolin and talc;
the natural molecules or the oils having filtrating properties of the UVB and/or UVA, such as carnauba wax, olive oil, karanja oil, usnic acid, propolis and cucumber extract.

When the UV filter is a mineral UV filter, being in the form of particles, said particles are advantageously covered with a hydrophobic coating, preferably based on methoxysilane, dimethicone, silica or alumina, so that these UV filters are solubilized in the oily phase, in other words that they are homogenously dispersed in the oily phase. This hydrophobic coating in known to those skilled in the art to increase the solubility in an oily phase of UV filters in mineral form.

Furthermore, these particles of mineral UV filters covered with a coating are advantageously dispersed in a mixture of solvents such as silicones, alkanes and vegetable oils in order to facilitate their incorporation into the oily phase. Such mixtures are perfectly known to those skilled in the art to disperse mineral UV filters, preferably covered with a hydrophobic coating, in an oily phase.

Preferably, the UV filter comprised in the nanocapsules in aqueous suspension according to the invention is titanium dioxide in nanometric form. Preferably, it is about titanium dioxide nanoparticles whose diameter of particles is comprised between 10 and 124 nm, and more preferably comprised between 10 and 110 nm. According to a variant of the invention, the diameter of particles is comprised between 15 and 124 nm.

For example, the oily phase will comprise from 0.1% to 70% by mass of UV filter, preferably from 0.1% to 20% of UV filter.

The oily phase also comprises at least a first surfactant, which may in particular act as a solubilizing agent of the UV filter. This first surfactant may be of the anionic, cationic, amphoteric or nonionic type.

The first surfactant may be in the form of an oil.

The first surfactant may have a HLB in the range of 3 to 6.

By way of example of the first surfactant, propylene glycol laurates, propylene glycol caprylates, polyglyceryl oleates, macrogolglycerides caprylocaproyles and sorbitan esters, may be mentioned.

According to one embodiment of the invention, the oily phase comprises from 2% to 50%, preferably from 2% to 6%, by mass of first surfactant.

According to a variant of the invention, the oily phase comprises from 4% to 50% by mass of first surfactant.

According to another variant of the invention, the oily phase comprises from 10% to 20% by mass of first surfactant.

Of course, the oily phase may contain one or more UV filter(s) and/or one or more hydrophobic polymer(s) and/or one or more first surfactant(s), meeting the criteria hereinabove.

The oily phase may, for example, be prepared by heating the hydrophobic polymer, at a temperature T1 greater than its melting temperature, then by adding the oil, then the UV filter. The first surfactant may be introduced at any stage of the preparation. The mixture may be done, in a different order where the set of components may be mixed all together.

It is also possible to heat oil to the temperature T1, and then to add the hydrophobic polymer in the liquid state, then the other components of the oily phase.

The obtained oily phase must be homogenous and, if necessary will be homogenized, for example with mechanical agitation.

The aqueous phase may also contain at least one second surfactant.

The second surfactant may be of the anionic, cationic, amphoteric or nonionic type.

According to a variant, the second surfactant has a HLB greater than or equal to 15, and is preferably selected from the neutral surfactants (for example polysorbates 20, 60 and 80; macrogol stearates; macrogol cetostearyl ethers; macrogol lauryl ethers, macrogol oleyl ethers, macrogol oleates; polyoxyl castor oil, hydrogenated polyoxyl castor oil).

According to one embodiment, the aqueous phase comprises from 0.1% to 12%, preferably from 5% to 10% by mass of second surfactant.

According to a variant of the invention, the aqueous phase comprises from 0.1% to 10% by mass of second surfactant.

According to another variant of the invention, the aqueous phase comprises from 0.1% to 5% by mass of second surfactant.

The aqueous phase may contain one or more second surfactant(s) meeting the criteria hereinabove.

In one embodiment of the invention, the aqueous phase further includes at least one hydrophilic polymer in the form of a hydrogel.

[symbol] Hydrophilic [symbol] polymer means a polymer soluble in aqueous solution. Polymer soluble in aqueous solution means a polymer which, when introduced in water at about 20° C., at a weight concentration equal to 1%, allows obtaining a solution which has a maximum light transmittance value, at a wavelength at which the polymer does not absorb, through a 1 cm thick sample, of at least 70%, preferably at least 80%.

[symbol] Hydrogel [symbol] means a homogenous gelatinous mixture forming a single phase containing water, and preferably including at least from 0.1 to 5% by mass of water, preferably 0.15 to 2% by mass of water.

By way of example, the hydrophilic polymer may be selected from synthetic cellulose derivatives, preferably from cellulose ethers such as methylcellulose, ethylcellulose, hydroxyethylcellulose, hydroxy propylcellulose, hydroxyethyl-methylcellulose, hydroxypropylmethylcellulose, methylethylcellulose and sodium carboxymethylcellulose and from poloxamers and polyvinyl alcohol.

According to one embodiment, the aqueous phase comprises from 10% to 40%, preferably from 25% to 35% by mass of hydrophilic polymer. The aqueous phase may contain one or more hydrophilic polymer(s) which meet the criteria hereinabove.

Presumably in this embodiment of the invention, the hydrophilic polymer forms a protective colloid around the nanocapsules, ensuring a greater stability of the colloidal suspension obtained and an improvement of the emulsification process.

The aqueous phase comprises, usually, from 60% to 90%, preferably from 65% to 75%, by mass of water or a mixture of water with one or more polar solvent(s). By way of example of polar solvent, ethanol, 1-propanol and 2-propanol may be mentioned.

The proportion of aqueous phase relative to the oily phase is variable.

When the aqueous phase includes a hydrophilic polymer, it may be used a mass ratio of hydrophobic polymer/hydrophilic polymer lower than or equal to 0.4.

The mixture between the oily phase and the aqueous phase may be carried out in various ways. It is possible to pour the oily phase into the aqueous phase or to mix the two phases by means of a mixer circuit in the form of [symbol] Y [symbol] , each of the two phases being brought in one of the two arm(s) of the [symbol] Y [symbol] . But, preferably, the two-phase mixture is carried out by adding the aqueous phase in the oily phase, under agitation. The oily phase is then maintained, during mixing, to a desired temperature T1 which is a temperature greater than the melting temperature of the hydrophobic polymer.

During mixing, the oily phase is at a temperature T1 greater than the melting temperature of the hydrophobic polymer. In one embodiment, it may be possible to use a temperature greater by 10° C. to 30° C. than the melting temperature of the hydrophobic polymer.

As mentioned hereinabove, it is essential that this temperature T1 is not too high to prevent any degradation of the other components used to prepare the nanocapsules according to the invention. Of course, those skilled in the art perfectly know the melting temperatures of the components of the oily phase and will be able to precisely choose the temperature T1 in order to obtain a homogeneous mixture without the degradation of the components of said oily phase. In this regard, the melting temperatures of the components are detailed in reference chemistry books, or even in the technical descriptions of the suppliers of the components of the oily phase.

If deemed necessary to obtain a perfectly homogeneous mixture, those skilled in the art will implement a mechanical agitation of the mixture. The parameters for carrying out the mechanical agitation are also perfectly within the capabilities of those skilled in the art.

At this temperature T1, the mixture composed of oil and of the first surfactant is miscible with the hydrophobic polymer, and the UV filter is also miscible, soluble or solubilized, optionally due to the first surfactant in the mixture composed of oil and of the first surfactant.

During mixing or just prior to mixing, the aqueous phase may be at room temperature, in particular at 20° C. or, according to a variant, the aqueous phase may also be heated.

In one embodiment, the aqueous phase is brought to a temperature T2 lower by 2° C. to 5° C. than the melting temperature of the hydrophobic polymer. When the aqueous phase includes a hydrophilic polymer, it is important to maintain, in the mixture conditions, the hydrogel character of the hydrophilic polymer.

According to an advantageous embodiment of the invention, the mixture of the oily phase and the aqueous phase is carried out under moderate agitation, preferably by using mechanical means operating at a rate within the range of 4000 to 16 000 rev/min and, according to a variant of the invention in the range of 6 000 to 8 000 rev/min. By way of example of suitable dispersing device to obtain an aqueous suspension of nanocapsules according to the invention, include a mechanical propeller shaft, blade or even anchor agitator, may be mentioned.

### DESCRIPTION OF THE UNIQUE FIGURE

FIG. 1 is a diagram of the measured and theoretical in vitro SPF values of 5 formulations according to the invention.

### EXPERIMENTAL PART

Different formulations was implemented to manufacture aqueous suspensions of nanocapsules according to the invention.

These formulations are detailed hereinafter with the percentages by mass (m/m) of each one of their components.

The formulations 1 to 8 hereinafter are compositions of aqueous suspensions of nanocapsules according to the present invention.

The formulations 1 to 8 was obtained according to the preparation process which was described hereinabove.

Formulation 1:
Lipid dispersion of titanium dioxide UV filter: 25%;
Carnauba wax: 5%;
Oleic Acid: 5%;
Polycaprolactone: 0.2%;
Sorbitan Oleate: 4.96%
Polysorbate 20: 7.04%
Distilled water: qsp 100%.

Formulation 2:
Lipid dispersion of titanium dioxide UV filter: 25%;
Carnauba wax: 5%;
Oleic Acid: 5%;
Polycaprolactone: 0.1%;
Sorbitan Oleate: 4.96%
Polysorbate 20: 7.04%;
Polysorbate 80 to 0.15% in distilled water: gsp 100%.

Formulation 3:
Lipid dispersion of titanium dioxide UV filter: 25%;
Oily mixture of oleoyl macrogol-6 glycerides, of oleoyl polyoxyl-6 glycerides, of apricot kernel oil and of polyethylene glycol esters and of stearic acid (hereinafter abbreviated PEG-6): 8%;
Mixture of polyglyceryl-3 dioleate, and of polyglyceryl-3 oleate: 5%;
Polycaprolactone: 0.4%;
25% Poloxamer 188 Gel in a 0.15% Polysorbate 80 solution in distilled water: qsp 100%.

Formulation 4:
Lipid dispersion of titanium dioxide UV filter: 27%;
Oily mixture of oleoyl macrogol-6 glycerides, of oleoyl polyoxyl-6 glycerides, of apricot kernel oil and of PEG-6 esters: 8%;
Mixture of polyglyceryl-3 dioleate and of polyglyceryl-3 oleate: 5%;
Polycaprolactone: 0.4%;
Fragrance: 1%;
Phenoxyethanol and ethylhexylglycerin: 1%;
27% Poloxamer 188 Gel in a 0.15% Polysorbate 80 solution in distilled water: qsp 100%.

Formulation 5:
Lipid dispersion of titanium dioxide UV filter: 27%;
Cerium oxide: 1%
Oily mixture of oleoyl macrogol-6 glycerides, of oleoyl polyoxyl-6 glycerides, of apricot kernel oil and of PEG-6 esters: 8%;
Mixture of polyglyceryl-3 dioleate and of polyglyceryl-3 oleate: 5%;
Polycaprolactone: 0.4%;
Polysorbate 20: 2%;
6% polyvinyl alcohol gel in 0.15% polysorbate 80 in distilled water qsp 100%.

Formulation 6:
Lipid dispersion of titanium dioxide UV filter: 18.29%;
Carnauba wax: 5%;
Oleic Acid: 5%;
Polycaprolactone: 0.2%;
Polysorbate 20: 8.22%;
Sorbitan oleate: 3.78%;
Distilled water: 59.51%.

Formulation 7:
Lipid dispersion of titanium dioxide UV filter: 6.25%;
Cerium oxide: 8%
Carnauba wax: 5%;
Oleic Acid: 5%;
Polycaprolactone: 0.2%;
Polysorbate 20: 9.72%;
Sorbitan oleate: 2.28%
Distilled water: 63.55%.

Formulation 8:
Carnauba wax: 5%;
Oleic acid: 5%;
Oxybenzone: 10%;
Polycaprolactone: 0.2%;
Sorbitan oleate: 0.93%;
Polysorbate 20: 11.07%;
Distilled water: qsp 100%.

Concerning the Composition of the Oily Phase:

The lipid dispersion of mineral titanium dioxide UV filters was a formulation of UV filter called [symbol] premix [symbol] . It comprised dispersed titanium dioxide nanoparticles. This lipid dispersion contained 49.8% by mass of titanium dioxide particles, whose diameter of particles was comprised between 15 and 124 nm, with an average diameter of 50 nm. The supplier of this dispersion of mineral titanium dioxide UV filters indicated an SPF of 2.5 to 3 by percentage of titanium dioxide incorporated into a sunscreen formulation.

Cerium oxide (CeO) is a mineral UV filter which mainly filters the UVA.

The oily mixture of oleoyl macrogol-6 glycerides, of oleoyl polyoxyl-6 glycerides, of apricot kernel oil and of PEG-6 esters is a mixture of solubilizing oils. In the formulations 3 to 5, it is about oil of the oily phase.

Polyglyceryl-3 dioleate and polyglyceryl-3 oleate are water-insoluble surfactants whose HLB are respectively 6 and 5. In the formulations 3 to 5, they were used as soluble surfactants in the oily phase.

Carnauba wax is a wax derived from leaves of a palm tree. It is solid at room temperature. It is used in the formulations 1, 2, 6 to 8 as an oil of the oily phase (in combination with oleic acid).

Oleic acid is an oil rich in fatty acids. It is used in the formulation 1, 2, 6 to 8 as an oil of the oily phase (and then in combination with carnauba wax).

Sorbitan oleate is a lipophilic emulsifying agent of vegetable origin. In the formulations 1, 2, 6 to 8, it was used as the first surfactant.

Concerning the composition of the aqueous phase:

Poloxamer 188 is a hydrophilic polymer, whose structure comprises ethylene oxide blocks (EO) and propylene oxide (PO) arranged according to the following tri-block structure: EO_{X}-PO_{Y}-EO_{X}, and which is in the form of a hydrogel. It is used in the formulations 3 and 4 for thickening and improving the final texture, as well as the stability of the aqueous suspension of nanocapsules according to the invention.

The polyvinyl alcohol gel present in the formulation 5 is a hydrophilic polymer in the form of hydrogel. It is used for thickening and improving the final texture and the stability of the aqueous suspension of nanocapsules according to the invention.

Polysorbate 80 and 20 are water-soluble surfactants with respective HLB 15 and 17. Within the framework of the invention, it is about surfactants soluble in the aqueous phase.

The mixture of phenoxyethanol and ethylhexylglycerin is a water-soluble excipient which was added as a preservative in the aqueous phase of the formulation 4.

The formulation 4 also contains a fragrance which was added in the aqueous phase.

The aqueous suspensions of nanocapsules of the formulations 1, 2, 6, 7 and 8 were carried out as follows:

Polycaprolactone which is a hydrophobic polymer was melted at about 95° C. in a beaker.

Carnauba wax, oleic acid and sorbitan oleate were mixed with melted polycaprolactone, with moderate mechanical agitation between 11 000 and 13 000 rev/min (rev/min is the abbreviation of [symbol] revolution per minute [symbol] ) by using a blade agitator.

The mineral or organic UV filters (namely oxybenzone for the formulation 8) detailed hereinabove were dispersed with the mixture of molten polycaprolactone with camauba wax, oleic acid and sorbitan oleate until obtaining a limpid mixture.

The aqueous phase was prepared by dispersing polysorbate 20 in distilled water with, if necessary for formulation 2, the addition of polysorbate 80.

The aqueous solution thus obtained was then heated at 90° C., to be dispersed, under moderate mechanical agitation between 13 000 and 16 000 rev/min using a blade agitator, in the mixture comprising molten polycaprolactone, mineral UV filters (or if necessary the organic UV filter), camauba wax, oleic acid and sorbitan oleate.

The formation of nanocapsules in aqueous suspension according to the invention was spontaneous under the effect of the aggregation of polycaprolactone in contact with the aqueous phase.

The aqueous suspensions of the nanocapsules of the formulations 3, 4 and 5 were carried out as follows:

Polycaprolactone was melted at about 95° C. in a beaker.

The oily mixture of oleoyl macrogol-6 glycerides, of oleoyl polyoxyl-6 glycerides, apricot kernel oil and PEG-6 esters and the oily mixture of polyglyceryl-3 dioleate and polyglyceryl-3 oleate were mixed together with molten polycaprolactone, under moderate mechanical agitation, namely between 11 000 and 13 000 rev/min, by using a blade agitator.

The mineral UV filters detailed hereinabove were dispersed with molten polycaprolactone and the oily mixture of oleoyl macrogol-6 glycerides, of oleoyl polyoxyl-6 glycerides, of apricot kernel oil, of PEG-6 esters, of polyglyceryl-3 dioleate, and of polyglyceryl-3 oleate until obtaining a limpid mixture.

The aqueous phase was prepared by dispersing the poloxamer gel in the case of the formulations 3 and 4, and the polyvinyl alcohol gel in the case of the formulation 5, in a polysorbate 80 solution under slow mechanical agitation, namely between 500 and 1 000 rev/min, if necessary, for the formulation 4 of the fragrance and the mixture of phenoxyethanol and ethylhexylglycerin and, for the formulation 5 of polysorbate 20.

The aqueous solution thus obtained was heated at 90° C. in order to be dispersed, under mechanical agitation between 11 000 and 13 000 rev/min, by using a blade agitator, in the mixture comprising molten polycaprolactone, the oily mixture of oleoyl macrogol-6 glycerides, of oleoyl polyoxyl-6 glycerides, of apricot kernel oil, of PEG-6 esters, of polyglyceryl-3 dioleate, and of polyglyceryl-3 oleate and the mineral UV filters.

The formation of nanocapsules in aqueous suspension according to the invention was spontaneous under the effect of the aggregation of polycaprolactone in contact with the aqueous phase.

For the formulations 1 to 8, it was determined:

1) the size of the nanocapsules (in nm);

2) the zeta potential (in mV);

3) the polydispersion index;

4) the critical wavelength (in nm).

More specifically, the size and the zeta potential of the nanocapsules was determined by using a particle size analyzer and a zetasizer (zeta nanosizer ZS, Malvern Instrument) respectively according to the principles of dynamic light scattering and electrophoresis by Doppler effect.

Table 1 hereinafter details the values obtained from these four parameters for each of the formulations 1 to 8.

TABLE 1
Values of the size, the polydispersion index, the zeta potential
and the critical wavelength of the formulations 1 to 8

| No of | | Polydis- | Zeta | Critical |
|---|---|---|---|---|
| formu- | Size | persion | Potentiel | wavelength |
| lation | (nm) | index | (mV) | (nm) |
| 1 | 148 ± 1 | 0.206 ± 0.007 | −39.0 ± 1.0 | 369.4 ± 1.0 |
| 2 | 175 ± 21 | 0.454 ± 0.047 | −38.4 ± 1.2 | 372.0 ± 0.0 |
| 3 | 637 ± 18 | 0.390 ± 0.052 | −27.6 ± 0.6 | 379.4 ± 0.9 |
| 4 | 440 ± 9 | 0.291 ± 0.022 | −34.2 ± 1.0 | 378.7 ± 0.4 |
| 5 | 323 ± 92 | 0.559 ± 0.038 | −30.2 ± 2.0 | 368.8 ± 0.8 |
| 6 | 128 ± 1 | 0.137 ± 0.016 | −28.6 ± 0.685 | 369.2 ± 0.4 |
| 7 | 194 ± 5 | 0.268 ± 0.027 | −25.5 ± 0.453 | 376.0 ± 0.0 |
| 8 | 768 ± 20 | 0.670 ± 0.01 | −32.7 ± 2.02 | 359.8 ± 0.8 |

According to the size values detailed in table 1 hereinabove, it is raised that the size of the nanocapsules may be comprised between about 100 nm and about 770 nm.

The polydispersion index of the suspensions of nanocapsules allows evaluating whether the nanocapsules are more or less dispersed in populations of different sizes.

When the polydispersion index is:
lower than 0.05: the suspension is called single-mode. There is a single size population.
comprised between 0.05 and 0.08: the suspension is almost single-mode.
comprised between 0.08 and 0.7: the suspension is of average polydispersity. In other words, there are several size populations of suspended particles.
greater than 0.7: the suspension is very polydispersed. In other words, there are several classes of sizes populations in the suspension.

In view of the values of the polydispersion index detailed in table 1, the suspensions of nanocapsules of the formulations 1 to 8 are all of average polydispersity.

The zeta potential measurement allows evaluating the load of the suspended nanocapsules in a solvent and thereby determining the stability of the aqueous suspension of nanocapsules.

It is estimated that a suspension of nanocapsules is stable, if its zeta potential is greater than 30 mV in absolute value.

There are two mechanisms which strongly affect the stability of a suspension: the steric repulsion and the electrostatic repulsion.

The suspension of nanocapsules according to the present invention comprises one or two polymer(s) and surfactants. These surfactants will largely influence the stability of these suspensions, since the nanocapsules will sterically repel one another and therefore this will inhibit the instability phenomena such as the flocculation or the coalescence of the suspended nanocapsules.

The suspension of nanocapsules according to the present invention already having a steric hindrance, it is estimated that a zeta potential of around 28 mV in absolute value indicates an acceptable stability.

In view of the values of the zeta potential detailed in table 1, the suspensions of nanocapsules of the formulations 1 to 8 all have an acceptable stability.

The critical wavelength corresponds to the wavelength below which the integral of the curve of the absorption spectrum beginning at 290 nm reaches 90% of the integral of 290 to 400 nm. It is known that for sunscreens having optimal efficiency, they must have a critical wavelength of at least about 370 nm. Given the values of the critical wavelengths detailed in table 1, it is raised that the formulations 1 to 7 all validate this required criteria regarding the critical wavelength. Indeed, the values of the critical wavelength of these formulations 1 to 7 are comprised between about 369 nm and 380 nm.

Concerning the formulation 8 which is the only formulation which comprises an organic UV filter (oxybenzone), the value of the critical wavelength of 359.8 nm is lower than the critical wavelength of the formulations 1 to 7 all of which comprise mineral UV filters. This is explained by the fact that oxybenzone is a UV filter which is known mainly for filtering UVB. The fact remains that the aqueous suspension of nanocapsules of the formulation 8 could be perfectly used in the formulation of a sunscreen.

Table 2 hereinafter details for each of the formulations 1 to 8:
The [symbol] theoretical [symbol] in vitro SPF which was estimated from the data provided by the supplier of the mineral or organic UV filters (for the case of the formulation 8) used in the formulations 1 to 8 which were in the form of (premix [symbol] mineral [symbol] or organic UV filters (in other words able to be directly incorporated into a sunscreen formulation).
The [symbol] measured [symbol] in vitro SPF was determined according to the guidelines of 2011 of the European federation of cosmetic industries: the [symbol] Cosmetics Europe Association [symbol] formerly called COLIPA.
The increase index of the SPF: the increase index of the SPF obtained due to the encapsulation of the UV filter in the nanocapsules according to the invention relative to the SPF of this same UV filter indicated by the supplier, namely [symbol] in free form [symbol] , that is to say non-encapsulated.

The increase index of the SPF is obtained according to the following formula II:

$$Increase Index of the S P F = \frac{( measured in vitro S P F ) - ( theoretical in vitro S P F )}{( theoretical in vitro S P F )} \times 100 \qquad ( II )$$

TABLE 2
Values of the theoretical and measured SPF and the increase
index of the SPF of the formulations 1 to 8

| No of | theoretical | measured | Increase |
|---|---|---|---|
| formu- | in vitro | in vitro | index of the |
| lation | SPF | SPF | SPF (%) |
| 1 | Between 30.7 and 36.9 | 66.2 | Between 79.4 and 115.3 |
| 2 | Between 30.7 and 36.9 | 49.1 | Between 33.1 and 59.9 |
| 3 | Between 30.7 and 36.9 | 46.3 | Between 25.5 and 50.6 |
| 4 | Between 33.2 and 39.8 | 52.1 | Between 30.8 and 56.9 |
| 5 | Between 33.2 and 39.8 | 51.8 | Between 30.1 and 55.8 |
| 6 | 25 | 62.1 | 150.0 |
| 7 | 8.5 | 29.1 | 243.5 |
| 8 | 11 | 19 | 73 |

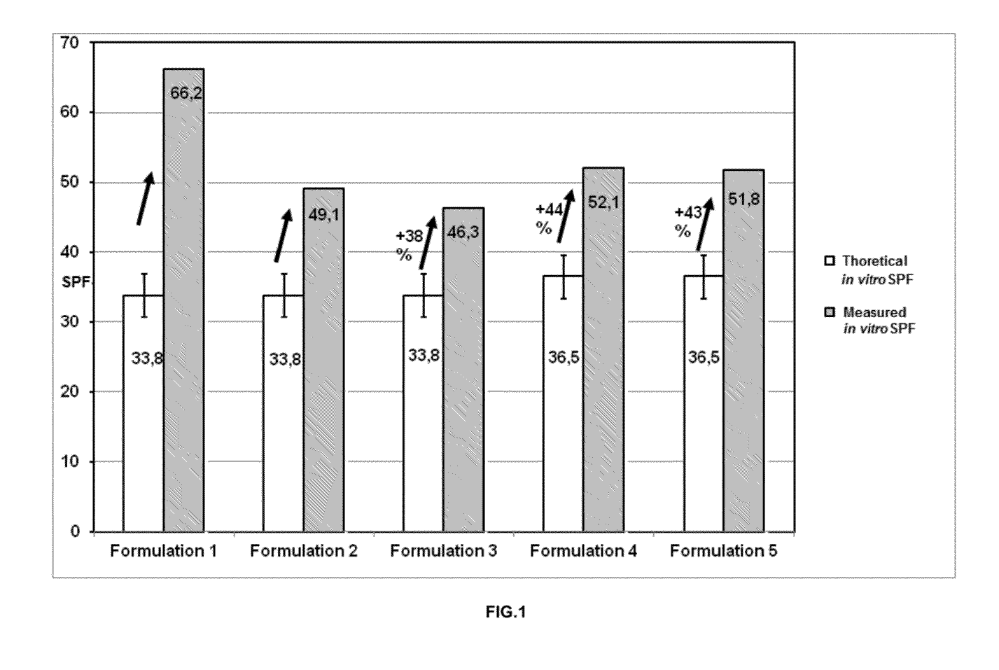

FIG. 1 is a histogram carried out from the values of the formulations 1 to 5 detailed in this table 2. More specifically, on this histogram FIGURE for the formulations 1 to 5:
the average value of the theoretical in vitro SPF, with the standard deviation bar;
the value of the measured in vitro SPF, and
the average value of the increase index of the SPF (expressed in percentage).

Table 2 and FIG. 1 also show that the formulations 1 to 8 according to the present invention exhibit an increase index of the SPF comprised between about 25% and 244%. This is quite remarkable and means that the encapsulation of the UV filters so as to obtain nanocapsules in an aqueous suspension according to the invention made it possible to very significantly increase the SPF of these UV filters.

Due to this very significant increase index of the SPF, it should be raised that these nanocapsules in an aqueous suspension according to the present invention are highly advantageous for their use in the sunscreen formulation, in particular sunscreen comprising mineral UV filters.

Indeed, in these sunscreen formulations, the UV filters concentration, in particular mineral UV filters concentration, may be lower than that contained in other sunscreen formulations incorporating these same mineral UV filters, and that while providing a sun protection in full compliance with the regulation in force.

Thus, due to this reduced amount of UV filters, in particular mineral UV filters (for example titanium dioxide), while ensuring a sun protection quite acceptable for the regulations, the sunscreen formulations comprising nanocapsules in aqueous suspension according to the invention will limit, or even remove, the aesthetic problem of white marks and/or the spreading difficulties of the sunscreen which could posed by the known sunscreen formulations containing these mineral UV filters.

It is easy to understand the advantages provided by the present invention with respect to the sunscreen formulation incorporating nanocapsules in aqueous suspension according to the invention.

Furthermore, the sunscreen formulations A and B hereinafter according to the present invention were prepared.

The compositions of these formulations A and B are detailed hereinafter. Among the components of these formulations A and B, there are the aqueous suspensions of nanocapsules of the formulations 6 and 7 which were described hereinabove.

Formulation A:
CeO particles: 5%;
lipid dispersion of ZnO: 1%;
Karanja oil: 5%; 25
the formulation 6: 67%;
the formulation 7: 18%;
di-glycerin: 1%;
fragrance: 1%;
Phenoxyethanol: 1%;
Sepineo P 600®: 1%.

Formulation B:
CeO particles: 3%;
lipid dispersion of ZnO: 2%; 35
Karanja oil: 8%;
formulation 6: 65%;
formulation 7: 18%;
di-glycerin: 1%;
fragrance: 1%;
Phenoxyethanol: 1%;
Sepineo P 600®: 1%.

In the formulations A and B:

The dispersion of zinc oxide (ZnO) was a lipid dispersion of UV filters called [symbol] premix [symbol] . It comprised 67% by mass of zinc oxide microparticles whose size was greater than 100 nm. The supplier of this zinc oxide dispersion indicated an SPF from 1 to 1.5 by percentage of zinc oxide incorporated into a sunscreen formulation.

Di-glycerin is a glycerin dimer. The trade name of the used product is [symbol] diglycerine S [symbol] .

Sepineo P 600® is a thickening, emulsifying and stabilizing polymer. More specifically, it is about a mixture of acrylamide, sodium acryloyldimethyl taurate copolymer, isohexadecane and Polysorbate 80.

Table 3 hereinafter details for each of the formulations A and B:
The massic percentages of the mineral UV filters which are titanium dioxide, zinc oxide and cerium oxide.
The [symbol] theoretical [symbol] in vivo SPF which was estimated from the data provided by the supplier of the mineral UV filters used in these formulations A and B, namely the zinc oxide dispersion, the titanium dioxide dispersion (formulation 6 and 7) and cerium oxide (formulation 7) and which were in the form of [symbol] premix [symbol] mineral UV filters—in other words which may be directly incorporated into a sunscreen formulation.

The [symbol] measured [symbol] in vivo SPF was determined according the guidelines of 2011 of the European federation of cosmetic industries: the [symbol] Cosmetics Europe Association [symbol] , formerly called COLIPA.
The increase index of the SPF: the increase index of the SPF obtained due to the encapsulation of the UV filter in the nanocapsules according to the invention relative to the SPF of this same UV filter indicated by the supplier, namely [symbol] in free form [symbol] , that is to say non-encapsulated.
The critical wavelength (nm).

TABLE 3
detailing the mass percentages of the mineral (TiO₂, ZnO
and CeO)UV filters, the theoretical in vivo SPF, the measured
in vivo SPF, the increase index of the SPF and the critical
wavelength of the formulations A and B.

| No of | | | | theo- | mea- | increase | Critical |
|---|---|---|---|---|---|---|---|
| for- | | | | retical | sured | index of | wave- |
| mula- | | ZnO | CeO | in vivo | in vivo | the SPF | length |
| tion | TiO _{2%} | % | % | SPF | SPF | (%) | (nm) |
| A | 6.58 | 0.68 | 6.44 | 19 | 37 | 95 | 376.3 ± 0.5 |
| B | 6.4 | 1.36 | 4.44 | 19 | 42 | 121 | 376.2 ± 0.4 |

According to table 3, it is raised that the critical wavelength values of the formulations A and B are greater than 370 nm. This demonstrates an optimal efficiency of sun protection of these sun sunscreen formulations.

Furthermore, it is raised that formulations A and B have an increase index of the SPF of respectively 95% and 121%. This demonstrates that the incorporation of nanocapsules in an aqueous suspension according to the invention in sunscreen formulations A and B allowed obtaining sunscreen formulations whose SPF was very significantly increased relative to the SPF of equivalent sunscreen formulations but in which the UV filters were not encapsulated.

The SPF values detailed in table 3 demonstrate the interest of incorporating, in sunscreen formulations, UV filters encapsulated in nanocapsules in aqueous suspension according to the invention.

The example detailed hereinafter is a comparative example between an aqueous suspension of nanocapsules according to the invention and a suspension of nanocapsules obtained according to the process described in the aforementioned application WO 2010/040194 A2.

More specifically, the SPF of two aqueous suspensions of nanocapsules loaded with 10% of chemical UV filter (oxybenzone) was compared:
the suspension 1, which exactly corresponds to the formulation 8 which was described hereinabove;
the suspension 2 which was obtained from the process described in the application WO 2010/040194 A2.

The suspension 2 had the following composition:
Oxybenzone: 10%;
Polycaprolactone: 0.19%;
Sorbitan oleate: 2%;
Karanja oil: 1%;
Acetone: 27 mL;
Polysorbate 80: 2%;
Distilled water: qsp 100%.

The suspension 2 was obtained from the preparation process described in the only example for preparation of nanocapsules of the application WO 2010/040194 A2, namely on page 17 of this international application.

The suspension 2 was obtained as follows:

An aqueous phase was prepared by dissolving Polysorbate 80 in distilled water.

An organic phase was prepared by mixing the sorbitan oleate, polycaprolactone, oxybenzone, Karanja oil in acetone, and this, so as all components of the organic phase (or in other words oily phase) are dissolved to obtain a homogeneous mixture.

Then, the organic phase was added to the aqueous phase and subjected the thus obtained mixture to an agitation to be fully homogenized.

The acetone was evaporated so as to obtain an aqueous suspension of nanocapsules.

Table 4 hereinafter details the theoretical and measured in vitro SPF, as well as the increase average index of the SPF of the suspensions 1 and 2.

TABLE 4
detailing the theoretical and measured in vitro SPF, as well as
the increase average index of the SPF of the suspensions 1 and 2

| | Average | | Average increase |
|---|---|---|---|
| | theoretical | Measured | index of the |
| Suspensions | in vitro SPF | in vitro SPF | SPF (in %) |
| Suspension 1 | 11 | 19 | 73 |
| Suspension 2 | 11 | 4 | 0 |

According to table 4, it is raised that the aqueous suspension 1 of nanocapsules according to the invention has an increase index of the SPF of 73%, and this, unlike the suspension 2 for which the increase index of the SPF is zero.

Thus, compared to aqueous suspensions of nanocapsules obtained according to another preparation process but with equivalent amounts of encapsulated UV filter, this comparative example demonstrates the interest of the aqueous suspensions of nanocapsules according to the invention which:

a) have an increase index of the SPF, and

b) do not require in their preparation process of the implementation of organic solvent in the oily phase and whose disadvantages was detailed hereinabove.

In other words, compared to aqueous suspensions of nanocapsules of the state of the art, in this case, the suspensions described in the application WO 2010/040194 A2, the aqueous suspensions of nanocapsules according to the invention exhibit not only more efficient sun protection properties, but also their preparation process is less constraining and faster, because it does not require a step for removing one of the components of the oily phase, namely the organic solvent (for example acetone) at the end of their preparation.

Furthermore, the aqueous suspensions of nanocapsules according to the invention, liberated from any organic solvent during the preparation of the oily phase, do not pose the risk of comprising this [symbol] impurity [symbol] consisting of the organic solvent. However, this risk remains for the aqueous suspensions of nanocapsules of the state of the art aforementioned, and despite all measures taken during the removal step of this organic solvent.

1. An aqueous suspension of nanocapsules which comprise an oily core wherein at least one UV filter is homogeneously dispersed and a polymeric envelope containing at least one hydrophobic polymer, said aqueous suspension of nanocapsules is likely to be obtained by a preparation process in which are mixed:
a) a first phase, called oily phase, which includes:
at least one hydrophobic polymer,
at least one oil,
at least one UV filter, and
at least a first surfactant,
this first phase being brought to a temperature T1 greater than the melting temperature of the hydrophobic polymer,
at this temperature T1, the hydrophobic polymer being miscible with the mixture of the first surfactant and the oil, and the UV filter being miscible, soluble or solubilized, in the mixture of the first surfactant and the oil;
b) a second phase, called aqueous phase, which comprises water and/or at least one polar solvent, and optionally at least a second surfactant,
so as to obtain the formation of nanocapsules in aqueous suspension.

2. The aqueous suspension of nanocapsules according to claim 1, wherein 1 the UV filter is chosen from the organic UV filters, the mineral UV filters or any compound having a filtering capability in the UVB and/or the UVA.

3. The aqueous suspension of nanocapsules according to claim 2, wherein the UV filter is selected from oxybenzone, sulisobenzone, dioxybenzone, ethyl anthranilate, avobenzone, terphatylidene, dicamphor sulfonic acid, bis-ethylhexyloxyphenol methoxyphenyl triazine, bis-ethylhexyloxyphenol, methoxyphenyl triazine, para-aminobenzoic acid, amyl p-dimethyl para-aminobenzoic acid, 2-ethoxyethyl-p-methoxycinnamate, digalloyl trioleate, ethyl 4-bishydroxypropylaminobenzone, 2-ethoxyethyl 2-cyano-3,3,diphenylacrylate, 2-ethylhexyl-p-methoxycinnamate, 2-ethylhexyl salicylate, glyceryl para-aminobenzoic acid, homo-methyl salicylate, dihydroxyacetone, octyl dimethyl para-aminobenzoic acid, 2-phenylbenzimidazole sulfonic acid and triethanolamine salicylate.

4. The aqueous suspension of nanocapsules according to claim 2, wherein the UV filter is chosen from titanium dioxide, zinc oxide, cerium oxide, kaolin and talc.

5. The aqueous suspension of nanocapsules according to claim 2, wherein the UV filter is selected from natural molecules or oils having filtration properties of UVB and/or UVA.

6. The aqueous suspension of nanocapsules according to claim 1, wherein the oily phase comprises from 0.1% to 70%.

7. The aqueous suspension of nanocapsules according to claim 1, wherein the oil is chosen from triglycerides, propylene glycol dicaprylocaprates, macrogolglycerides oleoyles, lauroyles and linoleoyles, vegetable oils, animal waxes and vegetable waxes.

8. The aqueous suspension of nanocapsules according to claim 1, wherein the oily phase comprises from 5% to 85%.

9. The aqueous suspension of nanocapsules according to claim 1, wherein the hydrophobic polymer is chosen from vinyl polymers, polyesters, polyamides, polyurethanes and polycarbonates.

10. The aqueous suspension of nanocapsules according to claim 1, wherein the oily phase comprises from 0.1% to 4%.

11. The aqueous suspension of nanocapsules according to claim 1, wherein the first surfactant is selected from propylene glycol laurates, propylene glycol caprylates, polyglyceryl oleates, caprylocaproyl macrogolglycerides, and sorbitan esters.

12. The aqueous suspension of nanocapsules according to claim 1, wherein the oily phase comprises from 2% to 50%.

13. The aqueous suspension of nanocapsules according to claim 1, wherein the second surfactant is selected from polysorbates 20, 60, 80, macrogol stearates, macrogol cetostearyl ethers, macrogol lauryl ethers, macrogol oleyl ethers, macrogol oleates, polyoxyl castor oil, and/or hydrogenated polyoxyl castor oil.

14. The aqueous suspension of nanocapsules according to claim 1, wherein the aqueous phase comprises from 0.1% to 12%.

15. The aqueous suspension of nanocapsules according to claim 1, wherein the nanocapsules have a diameter less than 1000 nm.

16. A sunscreen composition comprising at least one aqueous suspension of nanocapsules according to claim 1.

17. The sunscreen composition according to claim 16, wherein it is formulated in the form of a fluid emulsion, a thick emulsion, a gel, an oil, a stick or a lotion.
